(19) United States
(12) Patent Application Publication
(10) Pub. No.: US 2008/0281287 A1
(43) Pub. Date: Nov. 13, 2008

(54) SANITARY NAPKIN INCLUDING BODY-FACING PROTRUSIONS FOR PREVENTING SIDE LEAKAGE AND OBLIQUELY ARRANGED EMBOSSED CHANNELS

(76) Inventors: Ana Maria Elena R. Marcelo, Antipolo City (PH); Maria Socorro F. Medina, Cainta (PH)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/745,777

(22) Filed: May 8, 2007

(51) Int. Cl. A61F 13/15

(52) U.S. Cl. 604/383; 604/378; 604/385.01

(57) ABSTRACT

A sanitary napkin adapted to provide enhanced fluid handling characteristics and minimize side leakage, the sanitary napkin including a first embossing pattern including first and second spaced portions and a second embossing pattern including first and second spaced portions.

### FIELD OF INVENTION

The present invention generally relates to absorbent sanitary napkins and in particular to a sanitary napkin that effectively prevents side leakage while simultaneously providing superior wicking characteristics.

### BACKGROUND OF THE INVENTION

In order for a sanitary napkin to efficiently absorb a large amount of fluid during use it must effectively wick fluid throughout the absorbent structure of the napkin. Absent effective wicking properties menstrual fluid tends to pool in certain regions of the napkin as a result of which the full absorbent capacity of the napkin is not effectively utilized. On the other hand, napkins that exhibit particularly efficient wicking characteristics may exhibit other problems. In particular, such napkins may wick fluid to the longitudinal edge of the absorbent structure resulting in fluid being released from the longitudinal edge of the napkin, i.e. resulting in side leakage. Accordingly, there is a need to provide a sanitary napkin that efficiently wicks fluid yet at the same time effectively prevents side leakage.

### SUMMARY OF THE INVENTION

According to a first aspect of the invention, the present invention provides a sanitary napkin including a cover layer, a barrier layer, an absorbent core arranged between the cover layer and the barrier layer, the absorbent core having first and second longitudinally extending edges, a longitudinally extending centerline, a transversely extending centerline, a first and second longitudinal edge, a first and second transverse edge, a first end region and a second end region, a central region arranged between the first and second end regions, a first embossing pattern having a first portion and a second portion, the first portion located in the first end region and the second portion located in the second end region, the first and second portions being spaced from one another in a longitudinal direction of the napkin, each of the first and second portions including a plurality of channels, each one of the channels extending from one side of the longitudinally extending centerline to an opposite side of the longitudinally extending centerline, each one of the channels intersecting at least one other channel at an oblique angle relative thereto and each one of the channels extending across the longitudinally extending centerline at an oblique angle relative thereto, a second embossing pattern arranged in the central region, the second embossing pattern having a first portion located on a first side of the longitudinally extending centerline and a second portion located on a second side of the longitudinally extending centerline, the first and second portions being arranged in spaced relationship from one another in a transverse direction of the napkin, each of the first and second portions of the second embossing pattern being defined by a plurality of interconnected channels, the channels defining a plurality of body facing protrusions, an embossing-free zone located between the first and second portions of the second embossing pattern.

According to a second aspect of the invention, the present invention provides a sanitary napkin including a cover layer, a barrier layer, an absorbent core arranged between the cover layer and the barrier layer, the absorbent core having a first end second longitudinally extending edges, a longitudinally extending centerline, a transversely extending centerline, a first and second longitudinal edge, a first and second transverse edge, a first end region and a second end region, a central region arranged between the first end second end regions, a first embossing pattern having a first portion and a second portion, the first portion located in the first end region and the second portion located in the second end region, the first and second portions being spaced from one another in a longitudinal direction of the napkin, each of the first and second portions including a plurality of channels, each one of the channels extending from one side of the longitudinally extending centerline to an opposite side of the longitudinally extending centerline, each one of the channels intersecting at least one other channel at an oblique angle relative thereto and each one of the channels extending across the longitudinally extending centerline at an oblique angle relative thereto, each channel being substantially arcuate and having a length of about 3.5 cm to about 4.5 cm when measured along a path of the channel, each channel having a width from about 2 mm to about 3.5 mm, and each channel having a depth of about 1 to about 2 mm when measured from a top surface of the napkin, a second embossing pattern arranged in the central region, the second embossing pattern having a first portion located on a first side of the longitudinally extending centerline and a second portion located on a second side of the longitudinally extending centerline, the first and second portions being arranged in spaced relationship to one another in a transverse direction of the napkin, each of the first and second portions of the second embossing pattern being defined by a plurality of interconnected channels, the channels defining a plurality of body facing protrusions, each one of the body facing protrusions being completely surrounded by at least one of the interconnected channels, selected ones of the interconnected channels of the first portion of the second embossing pattern functioning to define a perimeter of the first portion of the second embossing pattern, selected ones of the interconnected channels of the second portion of the second embossing pattern functioning to define a perimeter of the second portion of the second embossing pattern, the perimeter of the first portion including an inner arcuate edge, an outer arcuate edge, first and second arcuate transverse edges interconnecting the inner and outer arcuate edges, the inner arcuate edge being spaced inwardly relative to the outer arcuate edge, the outer arcuate edge being spaced inwardly relative to the first longitudinally extending edge of the absorbent core by a distance of from about 5 mm to about 10 mm when measured along the transversely extending centerline of the napkin, the perimeter of the second portion including an inner arcuate edge, an outer arcuate edge, first and second arcuate transverse edges interconnecting the inner and outer arcuate edges, the inner arcuate edge being spaced inwardly relative to the outer arcuate edge, the outer arcuate edge being spaced inwardly relative to the second longitudinally extending edge of the absorbent core by a distance of from about 5 mm to about 10 mm when measured along the transversely extending centerline of the napkin, the inner arcuate edge of the first portion and the inner arcuate edge of the second portion being spaced from one another by a distance of from about 2 cm to about 3 cm as measured along the transversely extending centerline, an embossing-free zone located between the inner arcuate edge of the first portion and the inner arcuate edge of the second portion, the embossing-free zone located in an area of the napkin adapted to be arranged over the vaginal opening during use, the embossing-free zone having a width of from about 2 cm to about 3 cm as measured along the transversely extending centerline and a length of from about 5 cm to about 10 cm when measured along the longitudinally extending centerline, each one of the protrusions of the second embossing pattern having an apex that is from about 1 mm to above 2 mm above a bottom surface of a surrounding channel, a surface of the napkin in the embossing-free zone being located a distance of about 0.25 mm to about 2 mm above each of the apices of each of the protrusions, each one of the protrusions of the second embossing pattern having a surface area in the range of about 0.49 cm² to about 1.44 cm² and each one of the channels of the second embossing pattern having a width in the range of about 0.5 mm to about 3 mm, a first end channel located in the first end region and a second end channel located in the second end region, the first and second end channels being spaced from one another in a longitudinal direction of the napkin, each of the first and second end channels being substantially U-shaped, the first end channel being structured and arranged to substantially bound the first portion of the first embossing pattern and the second end channel being structured and arranged to substantially bound the second embossing pattern, each of the first and second end channels having a length of about 8 cm to about 12 cm as measured along a path of the respective channel, a width of about 2 to about 4 mm, and a depth of about 1 mm to about 2 mm as measured from a surface of the napkin.

### BRIEF DESCRIPTION OF THE DRAWINGS

Examples of embodiments of the present invention will now be described with reference to the drawings, in which:

FIG. 1 is a top plan view of a sanitary napkin in accordance with an embodiment of the present invention;

FIG. 2 is a bottom plan view of the sanitary napkin shown in FIG. 1;

FIG. 3 is a exploded perspective view of the napkin shown in FIG. 1 depicting the cover layer, barrier layer and absorbent core thereof;

FIG. 4 is an enlarged detail view of the portion of the napkin encircled by circle 4 in FIG. 1;

FIG. 5 is a cross sectional view taken along the transversely extending centerline 5-5 of the sanitary napkin shown in FIG. 1; and

FIG. 6 is a cross sectional view taken along a portion of longitudinally extending centerline 6-6 of the sanitary napkin shown in FIG. 1.

### DETAILED DESCRIPTION OF THE INVENTION

The present invention generally relates to disposable absorbent articles such as sanitary napkins, pantiliners, absorbent products for incontinence, and other disposable absorbent articles worn close to a wearer's body. As used herein, the term “sanitary napkin” refers to an article which is worn by females in an undergarment adjacent to the pudendal region and which is intended to absorb and contain various exudates which are discharged from the body (e.g., blood, menses, urine, and the like) and which is intended to be discarded after a single use (i.e., it is not intended to be laundered or otherwise restored or reused). Pantiliners are generally similar to sanitary napkins, except that they typically have lower capacity for absorbing fluids and are generally used to control non-menstrual discharges. Both sanitary napkins and pantiliners are typically attached or secured to a users undergarment and positioned between the undergarment and wearer's pudendal region. Adult incontinence articles, diapers, and interlabial devices are yet other disposable absorbent articles designed to manage various bodily exudates and may benefit from the embodiments of the invention described herein.

Absorbent articles according to the present invention provide superior fluid handling characteristics, and more specifically are particularly adept at preventing side leakage while at the same time providing wicking characteristics required to provide the superior fluid handling characteristics.

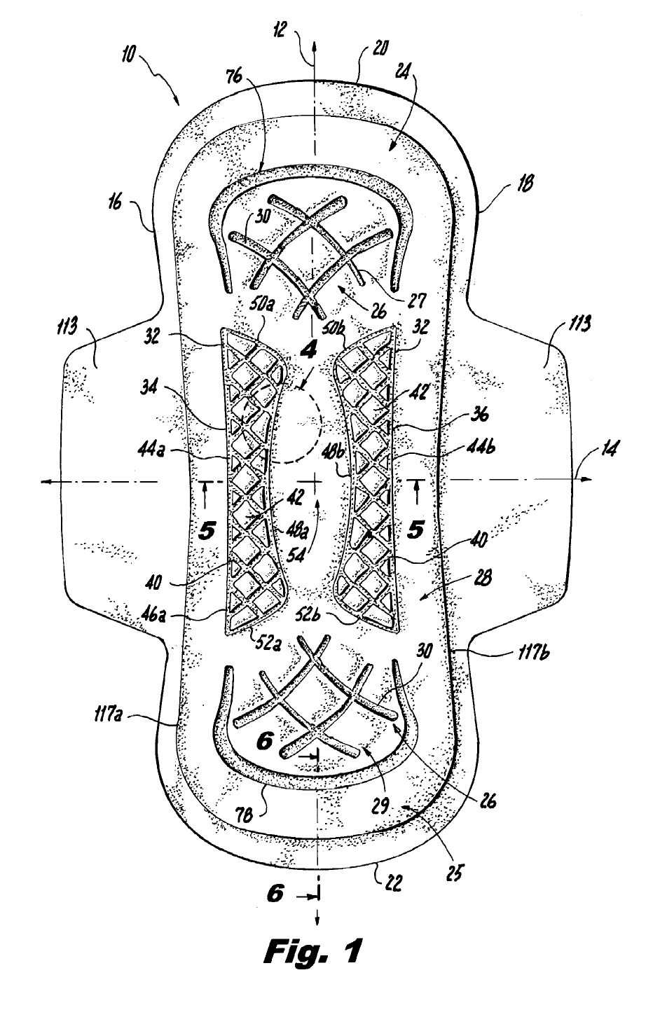

A specific embodiment of the sanitary napkin 10 according to the present invention is illustrated in FIG. 1. The sanitary napkin 10 includes a first a longitudinally extending centerline 12, a transversely extending centerline 14, a first longitudinal edge 16, a second longitudinal edge 18, a first transverse edge 20, a second transverse edge 22, a first end region 24, a second end region 25, and a central region 28 located between the first end second end regions, 24 and 25.

The napkin 10 further includes a first embossing pattern 26. The first embossing pattern 26 has a first portion 27 arranged in the first end region 24 and a second portion 29 located in the second end region 25, thus the first and second portions 27 and 29 are spaced from one another in the longitudinal direction of the napkin 10. Each of the first portion 27 and second portion 29 comprise a plurality of channels 30, each one of the channels 30 extending from one side of the napkin, across the longitudinally extending centerline 12, to the other side of the napkin. Each one of the channels 30 intersects at least one other channel 30 at an oblique angle relative thereto and each one of the channels 30 extends across the longitudinally extending centerline 12 at an oblique angle relative thereto.

Each of the channels 30 is substantially arcuate in shape and has a length in the range of about 2 cm to about 6 cm, more preferably from about 3.5 cm to about 4.5 cm, when measured along the path of the channel 30. Each of the channels 30 has a width in the range of from about 1 mm to about 10 mm, more preferably from about 2 mm to about 3.5 mm. Each of the channels 30 has a depth of about 0.5 mm to about 3 mm, more preferably about 1 mm to about 2 mm, when measured from a top surface of the napkin 10.

The napkin 10 further includes a second embossing pattern 32. The second embossing pattern 32 is located in the central region 28 of the napkin 10. The second embossing pattern 32 includes a first portion 34 located on a first side of the napkin, i.e. on one side of the longitudinally extending centerline 12, and a second portion 36 located on a second side of the napkin, i.e. on the other side of the longitudinally extending centerline 12.

Each of the first 34 and second portions 36 of the embossing pattern are defined by a plurality of interconnected fluid guiding channels 40, the fluid guiding channels defining therebetween a plurality of body facing protrusions 42. Each of the protrusions 42 are isolated, that is, each of the protrusions 42 is completely surrounded by at least one of the channels 40. The channels 40 that completely surround each protrusion 42 are generally regions of lesser height than the protrusion 42 and may be of higher compression or higher density than the protrusion 42 that is surrounded. Preferably the channels 40 of a respective portion, i.e. that is the channels 40 of the first portion 34 or the channels 40 of the second portion 36, are interconnected so as to form a network of continuously interconnected channels 40. The channels 40 also function to define a perimeter 44a of the first portion 34 and a perimeter 44b of the second portion 36.

The perimeter 44a of the first portion 34 includes an outer edge 46a that is substantially parallel to a longitudinally extending peripheral edge 117a of the absorbent core 103 of the napkin 10, and is spaced inwardly relative to the peripheral edge 117a. The perimeter 44a of the first portion 34 further includes an inner arcuate edge 48a that is arranged in inwardly spaced relationship to the outer edge 46a. The outer edge 46a and the inner arcuate edge 48a are interconnected by a first arcuate transverse edge 50a and a second arcuate transverse edge 52a. Preferably the outer edge 46a is spaced inwardly relative to the longitudinally extending peripheral edge 117a of the absorbent core 103 by a distance of about 2 mm to about 20 mm and more preferably by a distance of about 5 mm to about 10 mm, when measured along the transversely extending centerline 14 of the napkin 10.

Likewise, the perimeter 44b of the second portion 36 includes an outer edge 46b that is substantially parallel to a longitudinally extending peripheral edge 117b of the absorbent core 103, and spaced inwardly relative to the peripheral edge 117b. The perimeter 44b of the second portion 36 further includes an inner arcuate edge 48b that is arranged in inwardly spaced relationship to the outer edge 46b. The outer edge 46b and the inner arcuate edge 48b are interconnected by a first arcuate transverse edge 50b and a second arcuate transverse edge 52b. Preferably the outer edge 46b is spaced inwardly relative to the longitudinally extending peripheral edge 117b of the absorbent core by a distance of about 2 mm to about 20 mm and more preferably by a distance of about 5 mm to about 10 mm, when measured along the transversely extending centerline 14 of the napkin 10.

The first portion 34 and the second portion 36 are spaced from one another in the transverse direction, in particular the inner arcuate edge 48a of the first portion 34 is arranged in spaced relationship to the inner arcuate edge 48b of the second portion 36. The inner arcuate edge 48a and inner arcuate edge 48b function to substantially surround an embossing-free zone 54 zone located therebetween. Preferably the embossing-free zone 54 has a width as measured from the inner arcuate edge 48a to the inner arcuate edge 48b along the transversely extending centerline 14 in the range of about 1 cm to about 5 cm, and more preferably between about 2 cm and about 3 cm. The embossing-free zone 54 is located in an area of the napkin adapted arranged over the vaginal opening during use. The embossing-free zone 54 preferably has a length of about 3 cm to about 15 cm when measured along the longitudinally extending centerline 12, and more preferably from about 5 cm to about 10 cm. The embossing-free zone 54 is considered to end at its top and bottom at the beginning of the channels 30.

A projected area of the protrusions 42 (i.e., the sum of the area of each protrusion 42 within the first and second portions 34 and 36 as projected onto a two dimensional surface, such as is shown in FIG. 1) is greater than a projected area of the channels 40 (i.e., the sum of the area of each channel 40 within the first and second portions 34 and 36 as projected onto a two dimensional surface, such as is shown in FIG. 1). In a preferred embodiment of the invention, a ratio of the projected area of the protrusions 42 to the projected area of the channels 40 is less than 10. In a further preferred embodiment, the ratio is in a range from about 3 to about 7. Ratios above 10 are less desirable in that the channels 40 occupy too small a portion of the projected area of the sanitary napkin 10 relative to the protrusions 42, thus reducing fluid penetration in the first and second portions 34 and 36.

The channels 40 may be of uniform length or varying length. The plurality of channels 40 and the plurality of protrusions 42 may have various orientations.

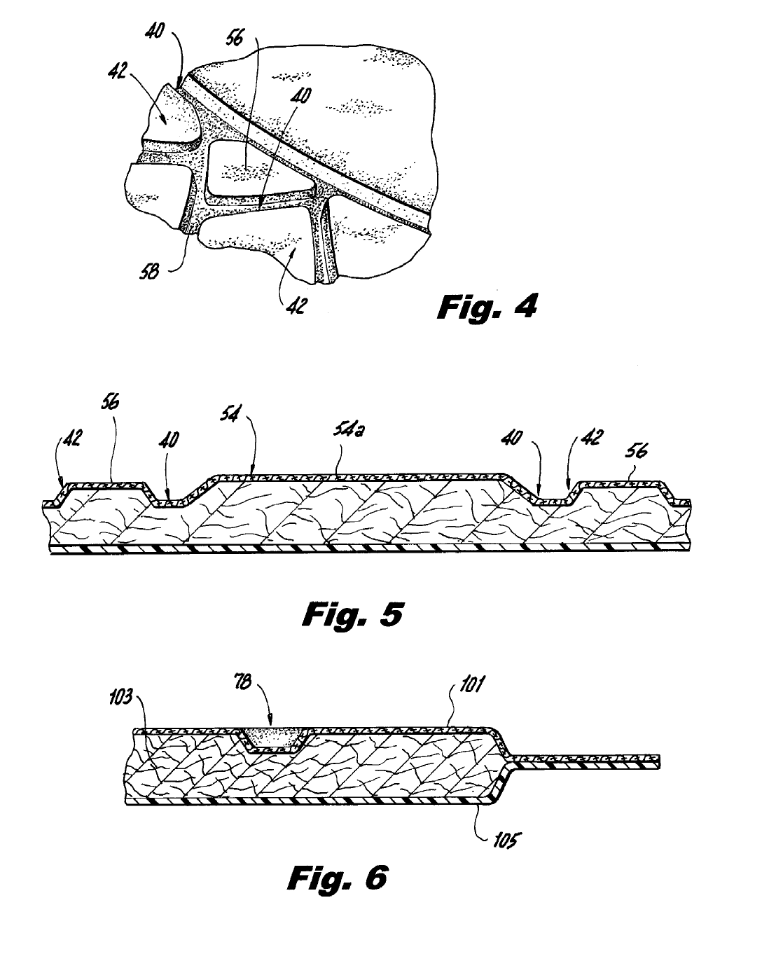

As shown in FIG. 4, each protrusions 42 has an apex 56 that is located about 0.5 mm to about 3 mm above bottom surface 58 of the surrounding channel 40, more preferably from about 1 mm to about 2 mm. Stated another way, each channel 40 preferably has a depth relative to an upper surface of the napkin of about 0.5 mm to about 3 mm, more preferably from about 1 mm to about 2 mm.

Due to the first and second embossed portions 34 and 36 located on either side of the embossing-free zone 54 the embossing-free zone 54 defines a slightly raised portion relative to apices 56. In particular, as best seen in FIG. 5, the surface of the napkin 54a in the area of the embossing-free zone 54 is preferably about 0.25 mm to about 2 mm higher than apex 56 more preferably about 0.5 mm to about 1 mm higher than the apex 56.

The channels 40 are generally shaped such that the bottom surface 58 is flat or concave (i.e., when the sanitary napkin 10 is laid flat, the lower surface 58 is not continuously sloping downward). Thus, the channels 40 are capable of guiding fluid within the first and second portions 34 and 36. Further the channels 40 and protrusions 42 are effective at preventing side leakage of fluid, i.e. leakage caused by movement of fluid in the transverse direction beyond the longitudinal edges of the absorbent system of the napkin.

The sanitary napkin 10 has a caliper that is the thickness of the sanitary napkin 10 as measured in an area outside the embossed areas of the napkin, i.e. outside the first and second embossing patterns 26 and 32. The caliper is measured when the sanitary napkin 10 is in a relaxed, uncompressed state, secured to prevent curling (such as by using 2 lb. weights arranged on the ends of the napkin to flatten the same), with any release papers removed, and is measured in a region that encompasses all material layers of the sanitary napkin 10. The caliper may be selected based upon desired technical properties desired of the sanitary napkin 10 (e.g. absorbency) or based upon consumer preference. In one embodiment of the invention, the caliper is less than about 5 mm.

In one embodiment of the invention, each protrusion 42 has a surface area in the range of about 0.09 cm² to about 4 cm² and more preferably in the range of about 0.49 cm² to about 1.44 cm². Each channel 40 preferably has a width in the range of about 0.25 mm to about 5 mm and more preferably from about 0.5 mm to about 3 mm. It has been found that the combination of the channels 40 and protrusions 42, having dimensions in the ranges described above, effectively transmit fluid within the first and second portions 34 and 36 and also are effective at preventing side leakage.

The sanitary napkin 10 may further optionally include a first end channel 76 and a second end channel 78. The first end channel 76 is located in the first end region 24 of the napkin 10 and the second end channel 78 is located in the second end region 26, thus the first end channel 76 is spaced from the second end channel 78 in the longitudinal direction of the napkin. Each of the first and second end channels 76 and 78 are substantially U-shaped and the first 76 and second 78 end channels function to substantially bound the first portion 27 and second portion 29 of the first embossing pattern 26.

“Substantially bound” as used herein means that the outermost point of the first end channel 76 in the longitudinal and transverse directions is located outside the outermost point of any one of the channels 30 located in the first portion 27, and further, none of the channels 30 intersect the first end channel 76. Likewise, the outermost point of the second end channel 78 in the longitudinal and transverse directions is located outside the outermost point of any one of the channels 30 located in the second portion 29, and further, none of the channels 30 intersect the second end channel 76.

Each of the first 76 and second end channels 78 preferably have a length of about 5 cm to about 26 cm and more preferably from about 8 cm to about 12 cm, as measured along the path of the channel, and a width of about 1 mm to about 10 and more preferably about 2 mm to about 4 mm. Each of the first 76 and second end channels 78 preferably depth of about 0.5 mm to about 3 mm, more preferably about 1 mm to about 2 mm as measured form a surface of the napkin.

### Construction of the Sanitary Napkin According to the Invention

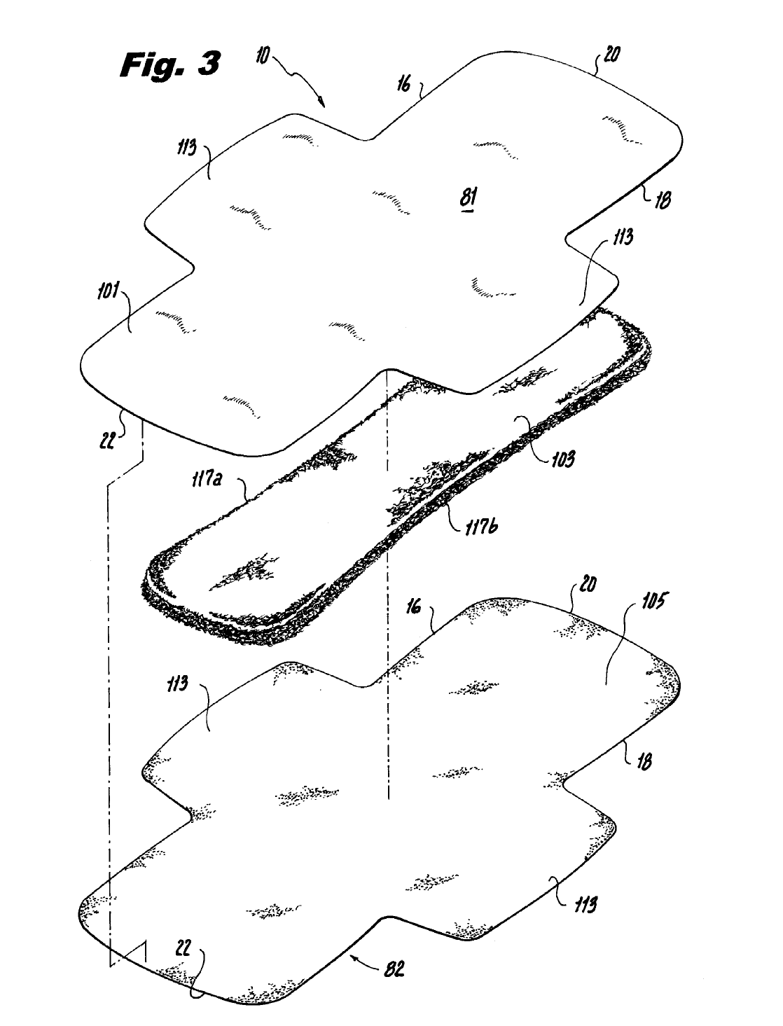

Referring to FIG. 3, the sanitary napkin 10 comprises a fluid-permeable cover layer 101, a liquid-impervious barrier layer 105 and an absorbent core 103 intermediate the fluid-permeable cover layer 101 and the liquid-impervious barrier layer 105. The cover layer 101 has a top surface that forms the body facing surface 81 of the sanitary napkin 10. The cover layer 101 is liquid permeable, and generally compliant, soft feeling, and non-irritating to the user's skin. It can be made from any of the materials conventional for this type of use. The cover layer 101 generally functions to transport fluid away from the wearer into the sanitary napkin 10. In this manner, fluid and moisture are removed from contacting the wearer, thus making the wearer feel dry and comfortable. Non-limiting examples of suitable materials that can be used as the cover layer 101 are woven and nonwoven fabrics formed from polyester, polypropylene, nylon, and/or rayon fibers or the topsheet may be an apertured thermo-plastic film and formed films. The cover layer 101 may optionally be treated with surfactant to manipulate the hydrophobicity/hydrophilicty thereof to facilitate optimal fluid transport properties. The fibers or other materials which make up the cover layer 101 should not collapse or lose their resiliency when subjected to body fluid. The cover layer 101 may be formed from, for example, staple fibers of polypropylene or other suitable materials. The fibers may be oriented by a carding process and thermally bonded via embossing. The basis weight of the cover layer 101 may range from about 10 grams per square meter (gsm) to about 40 gsm.

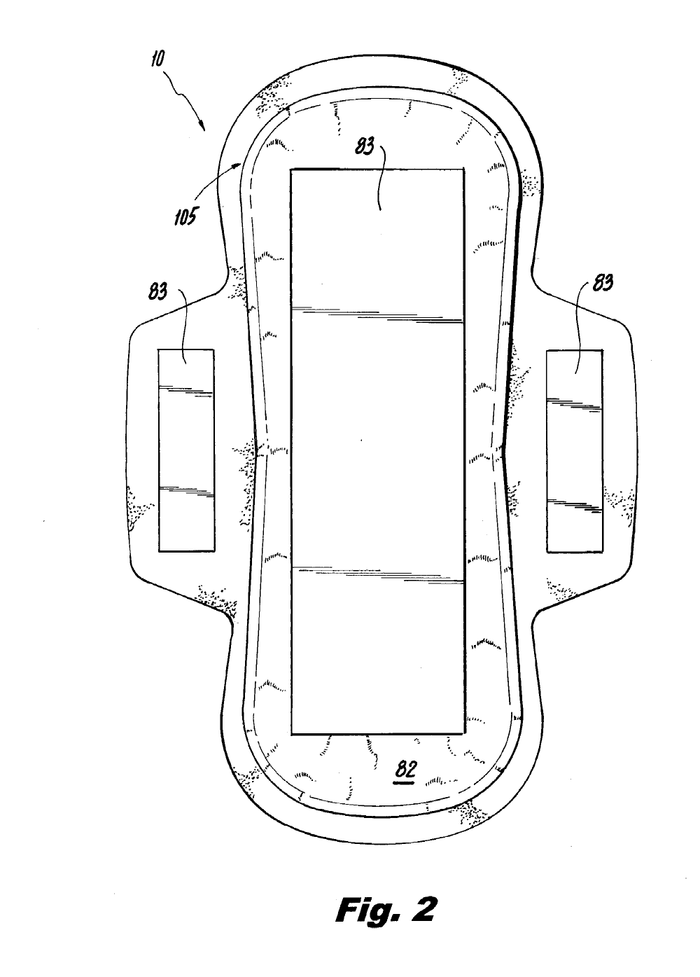

The barrier 105 has a bottom surface that forms the garment facing surface 82 of the napkin 10. The barrier layer 105 is impervious to liquids and, thus, prevents bodily fluid that may be present at the interface between the absorbent core 103 and the barrier layer 105 from soiling the clothing of the user. Suitable materials that may be incorporated into the barrier layer 105 include, for example, embossed or non-embossed polyethylene films, microporous films, and laminated tissue, among other materials. The garment facing surface 82 of the barrier layer 105 is preferably provided with garment attachment adhesive for securing the napkin 10 to an undergarment during use. The garment attachment adhesive is preferably covered with removable release paper 83 to protect the garment attachment adhesive prior to use as seen in FIG. 2.

The absorbent core 103 provides the means for absorbing bodily fluid. Bodily fluid moving inward or “down” from the cover layer 101 is conveyed to the absorbent core 103 which retains the bulk of the fluid until the sanitary napkin 1 is discarded. The absorbent core 103 has a high capacity for absorbing liquids and may be capable of maintaining the definition of the plurality of protrusions 42 and the plurality of channels 40 during the wearing of the sanitary napkin 1.

Examples of material that may be used in the construction of the absorbent core 103 include, for example, cellulosic fibers (preferably wood pulp, but cotton, flax and peat moss are acceptable), synthetic fibers, superabsorbent polymers (SAP) or superabsorbent fibers, as well organic binders and other materials known to the art of manufacturing absorbent core materials. The relative proportion of these materials may be varied to achieve sufficient absorbency, compressibility, and processibility. In one non-limiting example, the absorbent core 103 comprises from about 40 weight percent to about 95 weight percent cellulosic fibers, and from about 5 weight percent to about 60 weight percent superabsorbent polymer.

The absorbent core 103 may include any superabsorbent polymer (SAP). For the purposes of the present invention, the term “superabsorbent polymer” (or “SAP”) refers to materials which are capable of absorbing and retaining at least about 10 times their weight in body fluids under a 0.5 psi pressure. The superabsorbent polymer particles of the invention may be inorganic or organic crosslinked hydrophilic polymers, such as polyvinyl alcohols, polyethylene oxides, crosslinked starches, guar gum, xanthan gum, and other material known to the art of absorbent article manufacture.

The sanitary napkin 10 may optionally further include a transfer layer (not shown) and, if present, the transfer layer generally positioned directly underneath the cover layer 101, and the transfer layer directly contacts the absorbent core. The transfer layer provides the means of receiving body fluid from the fluid-pervious cover layer 101 and holding it until the absorbent core has an opportunity to absorb it. The transfer layer is, preferably, more dense than the fluid-pervious cover layer 101 and has a larger proportion of smaller pores than does the latter. These attributes allow the transfer layer to contain body fluid and hold it away from the outer side of the fluid-pervious cover layer 101, thereby preventing the fluid from re-wetting the fluid-pervious cover layer 101 and its surface. However, the transfer layer is preferably not so dense as to prevent the passage of the fluid through the transfer layer and into the underlying absorbent core.

The optional transfer layer may comprise various materials, including, for example, cellulose fibers such as from wood pulp, single component or bicomponent fibers that include thermoplastic materials (such as polyester, polypropylene, polyethylene, among others) in fiber or other forms, rayon, organic binders (such as copolymers of vinyl, acrylic and/or other monomers that may be coated onto thermoplastic fibers or otherwise incorporated into the transfer layer) among other materials known to the art. The transfer layer may, for example, have a basis weight in a range from about 20 grams per square meter (gsm) to about 120 gsm.

While the various material layers (cover, absorbent system, barrier) are described as separate layers, it is within the scope of the invention that one or more of these layers may be formed or integrated together and may actually not be discrete material layers, but rather a unitary layer possessing multiple functional properties.

The channels 30, channels 40 and protrusions 42, and channels 76 and 78, preferably extend into additional material layers of the sanitary napkin 10. For example, in the embodiment of the invention shown in FIG. 1 and FIG. 3, the channels 30, channels 40 and protrusions 42 are formed through the cover layer 101 and the absorbent core 103. By “formed through,” the absorbent core 103, it is meant that if one were to follow a top surface absorbent core 103, one would find a plurality of channels 30, channels 40 and protrusions 42 that correspond, register, with or align with the channels 30, channels 40 and protrusions 42 of the cover layer 101. As such, the cover layer 101 follows the contours in the top surface of the absorbent core 103 with no appreciable macroscopic voids present between the two layers.

Referring again to FIG. 3, the absorbent core 103 may be confined to a laterally central region of the sanitary napkin 10 such that the cover layer 101 and barrier 105 extend beyond a peripheral edge 117 of the absorbent core 103. Alternatively, the absorbent core 103 may extend laterally into the flap regions 113. As best seen in FIG. 6, the cover layer 101 and the barrier layer 105 are joined around the entire periphery of the sanitary napkin 10. The cover layer 101 and the barrier layer 105 can be joined by any means commonly used in the art for this purpose such as by gluing, crimping, or heat-sealing. Additional securement of the layers 101, 105 and 103 may be achieved by laminating one or more of these layers together.

The sanitary napkin 10 may be made using various conventional processes known to those of skill in the art, such as, for example, an embossing process in which one or more material layers of the sanitary napkin 10 are subject to mechanical and thermal energy to form channels 30, channels 40 and protrusions 42, and channels 76 and 78.

In accordance with one aspect of the invention, a method of making the sanitary napkin 10 comprises providing a body-faceable, liquid-pervious cover layer 101 having a top surface, a garment-faceable, liquid-impervious barrier layer 105, and an absorbent core 103. The cover layer 101 is arranged on top of the absorbent core 103 and the layers are embossed to provide a plurality of channels 30, channels 40 and protrusions 42, channels 76 and 78 on the surface of the cover layer 101 and the channels 30, channels 40 and protrusions 42, and channels 76 and 78 are formed through the absorbent core 103. The barrier layer 105 is then sealed to the cover layer 101 about a perimeter thereof using heat and pressure.

### EXAMPLES

### Inventive Example #1

A thin sanitary napkin was constructed including a 22 gsm hot through air bonded cover made from polypropylene/polyethylene bicomponent fibers, a pulp core including 5.5 g of fluff pulp and 0.25 g of superabsorbent, and a 23 gsm polyethylene barrier layer. The sanitary napkin was embossed to include the embossing pattern depicted in FIG. 1 and the sanitary napkin was embossed such that the pattern was formed through the cover and the core but not the barrier.

### Procedure for Measuring Side Leakage

To illustrate the improved fluid handling characteristics of the sanitary napkin of Example #1 described above, and in particular the ability of such a napkin to prevent side leakage, the sanitary napkin of Example #1 was tested according to the test procedure described below. For comparative purposes commercially available napkins were also tested and the results of the above procedures are summarized in Table #1 provided below.

A sanitary napkin according to Example #1 was tested according to the following procedure.
(1) 7.0 ml of a synthetic menstrual fluid was placed in a funnel;
(2) A petri dish was weight and placed underneath the napkin;
(3) The tip of the funnel was arranged at the center of the napkin and was arranged such that the tip of the funnel was spaced 10 mm from the top surface of the napkin;
(4) The valve of the funnel was fully opened;
(5) The pertri dish was observed to determine if there was any leakage from, if any fluid was found in the petri dish the test was considered “failed”;
(6) The petri dish was weighed to determine the amount of leakage;
(7) The above described test was repeated for thirty samples;
(8) The percentage of failure was calculated by dividing the number of total failed samples by the total number of samples tested (i.e. 30 samples).

The above described test was conducted on inventive Example #1 and on the following commercially available products, Modess Maxi Cottony Soft Cover, Polyboat, Kotex Soft and Smooth Maxi, Whisper Regular Flow, and Sofy Body Fit Slim.

TABLE #1

| | | Modess Maxi | | Kotex Soft | | |
|---|---|---|---|---|---|---|
| | Inventive | Cottony Soft | | and Smooth | Whisper | Sofy Body |
| Sample | Example #1 | Cover | Polyboat | Maxi | Regular Flow | Fit Slim |
| 1 | 0.00 | 2.90 | 4.91 | 4.10 | 0.00 | 0.00 |
| 2 | 0.00 | 4.16 | 4.03 | 4.02 | 1.23 | 0.00 |
| 3 | 0.00 | 2.03 | 4.27 | 4.33 | 0.00 | 3.64 |
| 4 | 0.00 | 4.09 | 4.77 | 1.04 | 0.00 | 2.75 |
| 5 | 0.00 | 3.55 | 5.32 | 2.94 | 0.00 | 0.00 |
| 6 | 3.02 | 1.46 | 3.16 | 4.53 | 3.54 | 0.00 |
| 7 | 0.00 | 0.00 | 6.15 | 4.06 | 2.85 | 2.29 |
| 8 | 0.00 | 0.00 | 3.69 | 1.38 | 0.00 | 2.80 |
| 9 | 0.00 | 4.27 | 5.79 | 1.47 | 0.00 | 0.00 |
| 10 | 0.00 | 2.32 | 4.48 | 2.94 | 0.00 | 0.75 |
| 11 | 0.00 | 0.00 | 4.62 | 3.95 | 0.00 | 0.00 |
| 12 | 0.00 | 0.00 | 5.38 | 1.49 | 0.00 | 2.57 |
| 13 | 0.00 | 3.63 | 5.21 | 0.30 | 3.79 | 1.00 |
| 14 | 0.00 | 4.34 | 4.02 | 2.67 | 0.00 | 4.01 |
| 15 | 0.00 | 0.25 | 3.70 | 2.29 | 0.00 | 0.00 |
| 16 | 0.00 | 4.36 | 4.91 | 4.84 | 0.00 | 0.00 |
| 17 | 1.93 | 0.00 | 2.75 | 4.39 | 0.71 | 0.00 |
| 18 | 0.00 | 0.00 | 5.03 | 4.15 | 0.00 | 0.00 |
| 19 | 0.00 | 0.00 | 5.69 | 4.41 | 2.06 | 2.95 |
| 20 | 0.00 | 0.00 | 4.04 | 1.94 | 0.00 | 2.92 |
| 21 | 0.00 | 0.56 | 5.17 | 4.04 | 2.06 | 2.99 |
| 22 | 0.00 | 0.85 | 4.96 | 5.45 | 0.00 | 4.50 |
| 23 | 0.00 | 1.79 | 4.15 | 4.60 | 2.44 | 0.00 |
| 24 | 0.00 | 1.48 | 5.35 | 4.14 | 2.73 | 0.00 |
| 25 | 0.00 | 0.00 | 4.91 | 4.34 | 2.70 | 2.57 |
| 26 | 0.00 | 0.00 | 4.66 | 5.06 | 0.00 | 0.00 |
| 27 | 0.00 | 1.28 | 3.91 | 3.70 | 0.00 | 4.21 |
| 28 | 0.00 | 0.00 | 5.58 | 5.20 | 0.00 | 1.84 |
| 29 | 0.00 | 0.00 | 5.20 | 2.98 | 0.00 | 0.44 |
| 30 | 0.00 | 0.36 | 5.07 | 3.65 | 3.88 | 0.00 |
| Total Number | 30 | 30 | 30 | 30 | 30 | 30 |
| of Samples | | | | | | |
| Leakage | 2 | 18 | 30 | 30 | 11 | 16 |
| Occurrence | | | | | | |
| Percentage of | 6.67 | 60.00 | 100.00 | 100.00 | 36.67 | 53.33 |
| leakage | | | | | | |
| occurrence | | | | | | |

As illustrated by the above test the napkin according to the present invention exhibited superior fluid handling characteristics and in particular significantly reduced the occurrence of side leakage.

We claim:

1. A sanitary napkin comprising:
a cover layer;
a barrier layer;
an absorbent core arranged between the cover layer and the barrier layer, the absorbent core having first and second longitudinally extending edges;
a longitudinally extending centerline;
a transversely extending centerline;
a first and second longitudinal edge;
a first and second transverse edge;
a first end region and a second end region,
a central region arranged between the first and second end regions;
a first embossing pattern having a first portion and a second portion, the first portion located in the first end region and the second portion located in the second end region, the first and second portions being spaced from one another in a longitudinal direction of the napkin, each of the first and second portions comprising a plurality of channels, each one of the channels extending from one side of the longitudinally extending centerline to an opposite side of the longitudinally extending centerline, each one of the channels intersecting at least another channel at an oblique angle relative thereto and each one of the channels, extending across the longitudinally extending centerline at an oblique angle relative thereto;
a second embossing pattern arranged in the central region, the second embossing pattern having a first portion located on a first side of the longitudinally extending centerline and a second portion located on a second side of the longitudinally extending centerline, the first and second portions being arranged in spaced relationship from one another in a transverse direction of the napkin, each of the first and second portions of the second embossing pattern being defined by a plurality of interconnected channels, the channels defining a plurality of body facing protrusions;
an embossing-free zone located between first and second portions of the second embossing pattern.

2. The sanitary napkin according to claim 1, wherein each channel of the first embossing pattern is substantially arcuate.

3. The sanitary napkin according to claim 1, wherein each channel of the first embossing pattern has a length of about 2.0 cm to about 6.0 cm when measured along a path of the channel.

4. The sanitary napkin according to claim 3, wherein each channel of the first embossing pattern has a width from about 2 mm to about 3.5 mm,

5. The sanitary napkin according to claim 4, wherein each channel of the first embossing pattern has a depth of about 1 to about 2 mm when measured from a top surface of the napkin.

6. The sanitary napkin according to claim 1, wherein selected ones of the interconnected channels of the first portion of the second embossing pattern function to define a perimeter of the first portion of the second embossing pattern, and selected ones of the interconnected channels of the second portion of the second embossing pattern function to define a perimeter of the second portion of the second embossing pattern.

7. The sanitary napkin according to claim 6, wherein the perimeter of the first portion includes an inner arcuate edge, an outer arcuate edge, first and second arcuate transverse edges interconnecting the inner and outer arcuate edges.

8. The sanitary napkin according to claim 7, wherein the inner arcuate edge is spaced inwardly relative to the outer arcuate edge and the outer arcuate edge is spaced inwardly relative to the first longitudinally extending edge of the absorbent core.

9. The sanitary napkin according to claim 8, wherein the outer arcuate edge is spaced inwardly relative to the first longitudinally extending edge of the absorbent core by a distance of from about 5 mm to about 10 mm when measured along the transversely extending centerline of the napkin.

10. The sanitary napkin according to claim 6, wherein the perimeter of the second portion includes an inner arcuate edge, an outer arcuate edge, and first and second arcuate transverse edges interconnecting the inner and outer arcuate edges.

11. The sanitary napkin according to claim 10, wherein the inner arcuate edge is spaced inwardly relative to the outer arcuate edge and the outer arcuate edge is spaced inwardly relative to the second longitudinally extending edge of the absorbent core.

12. The sanitary napkin according to claim 11, wherein outer arcuate edge is spaced inwardly relative to the second longitudinally extending edge of the absorbent core by a distance of from about 5 mm to about 10 mm when measured along the transversely extending centerline of the napkin.

13. The sanitary napkin according to claim 6, wherein an inner arcuate edge of the first portion and an inner arcuate edge of the second portion are spaced from one another by a distance of from about 2 cm to about 3 cm as measured along the transversely extending centerline.

14. The sanitary napkin according to claim 1, wherein the embossing-free zone located in an area of the napkin adapted to be arranged over the vaginal opening during use.

15. The sanitary napkin according to claim 14, wherein the embossing-free zone has a width of from about 2 cm to about 3 cm as measured along the transversely extending centerline.

16. The sanitary napkin according to claim 15, wherein the embossing-free zone has a length of from about 5 cm to about 10 cm when measured along the longitudinally extending centerline.

17. The sanitary napkin according to claim 1, further comprising a first end channel located in the first end region and a second end channel located in the second end region.

18. The sanitary napkin according to claim 16, wherein the first and second end channels are spaced from one another in a longitudinal direction of the napkin, the first end channel being structured and arranged to substantially bound the first portion of the first embossing pattern and the second end channel being structured and arranged to substantially bound the second embossing pattern.

19. A sanitary napkin comprising:
a cover layer;
a barrier layer;
an absorbent core arranged between the cover layer and the barrier layer, the absorbent core having a first end second longitudinally extending edges;
a longitudinally extending centerline;
a transversely extending centerline;
a first and second longitudinal edge;
a first and second transverse edge;
a first end region and a second end region,
a central region arranged between the first end second end regions;
a first embossing pattern having a first portion and a second portion, the first portion located in the first end region and the second portion located in the second end region, the first and second portions being spaced from one another in a longitudinal direction of the napkin, each of the first and second portions comprising a plurality of channels, each one of the channels extending from one side of the longitudinally extending centerline to an opposite side of the longitudinally extending centerline, each one of the channels intersecting at least one other channel at an oblique angle relative thereto and each one of the channels extending across the longitudinally extending centerline at an oblique angle relative thereto, each channel being substantially arcuate and having a length of about 3.5 cm to about 4.5 cm when measured along a path of the channel, each channel having a width from about 2 mm to about 3.5 mm, and each channel a depth of about 1 to about 2 mm when measured from a top surface of the napkin;
a second embossing pattern arranged in the central region, the second embossing pattern having a first portion located on a first side of the longitudinally extending centerline and a second portion located on a second side of the longitudinally extending centerline, the first and second portions being arranged in spaced relationship from one another in a transverse direction of the napkin, each of the first and second portions of the second embossing pattern being defined by a plurality of interconnected channels, the channels defining a plurality of body facing protrusions, each one of the body facing protrusions being completely surrounded by at least one of the interconnected channels, selected ones of the interconnected channels of the first portion of the second embossing pattern functioning to define a perimeter of the first portion of the second embossing pattern, selected ones of the interconnected channels of the second portion of the second embossing pattern functioning to define a perimeter of the second portion of the second embossing pattern, the perimeter of the first portion including an inner arcuate edge, an outer arcuate edge, first and second arcuate transverse edges interconnecting the inner and outer arcuate edges, the inner arcuate edge being spaced inwardly relative to the outer arcuate edge, the outer arcuate edge being spaced inwardly relative to the first longitudinally extending edge of the absorbent core by a distance of from about 5 mm to about 10 mm when measured along the transversely extending centerline of the napkin, the perimeter of the second portion including an inner arcuate edge, an outer arcuate edge, first and second arcuate transverse edges interconnecting the inner and outer arcuate edges, the inner arcuate edge being spaced inwardly relative to the outer arcuate edge, the outer arcuate edge being spaced inwardly relative to the second longitudinally extending edge of the absorbent core by a distance of from about 5 mm to about 10 mm when measured along the transversely extending centerline of the napkin, the inner arcuate edge of the first portion and the inner arcuate edge of the second portion being spaced from one another by a distance of from about 2 cm to about 3 cm as measured along the transversely extending centerline;
an embossing-free zone located between the inner arcuate edge of the first portion and the inner arcuate edge of the second portion, the embossing-free zone located in an area of the napkin adapted to be arranged over the vaginal opening during use, the embossing-free zone having a width of from about 2 cm to about 3 cm as measured along the transversely extending centerline and a length of from about 5 cm to about 10 cm when measured along the longitudinally extending centerline;
each one of the protrusions of the second embossing pattern having an apex that is from about 1 mm to above 2 mm above a bottom surface of a surrounding channel;
a surface of the napkin in the embossing-free zone being located a distance of about 0.25 mm to about 2 mm above each of the apices of each of the protrusions;
each one of the protrusions of the second embossing pattern having a surface area in the range of about 0.49 cm² to about 1.44 cm² and each one of the channels of the second embossing pattern having a width in the range of about 0.5 mm to about 3 mm;
a first end channel located in the first end region and a second end channel located in the second end region, the first and second end channels being spaced from one another in a longitudinal direction of the napkin, each of the first and second end channels being substantially U-shaped, the first end channel being structured and arranged to substantially bound the first portion of the first embossing pattern and the second end channel being structured and arranged to substantially bound the second embossing pattern, each of the first and second end channels having a length of about 8 cm to about 12 cm as measured along a path of the respective channel, a width of about 2 to about 4 mm, and a depth of about 1 mm to about 2 mm as measured from a surface of the napkin.
